(19) United States
(12) Patent Application Publication
(10) Pub. No.: US 2004/0100617 A1
(43) Pub. Date: May 27, 2004

(54) Apparatus for interactive optometry

(76) Inventor: Mordechai Abitbol, Jerusalem (IL)

Correspondence Address: FLIESLER MEYER, LLP, FOUR EMBARCADERO CENTER, SUITE 400, SAN FRANCISCO, CA, 94111, US

(21) Appl. No.: 10/343,381

(22) PCT Filed: Jul. 29, 2001
(86) PCT No.: PCT/IL01/00698

(30) Foreign Application Priority Data
Aug. 1, 2000 (IL) 137635

(51) Int. Cl. A61B 3/10

(52) U.S. Cl. 351/205

(57) ABSTRACT

A system for determining with essentially continuous variability, wave aberrations originating in an eye of a subject, according to the visual perception of said subject.

### FIELD OF THE INVENTION

The present invention relates to the field of optometric measurements, especially those performed in an interactive manner with the subject.

### BACKGROUND OF THE INVENTION

The most widely used method of optometric measurement for determining the specifications of vision correction lenses is by means of the familiar trial and error process, whereby the subject whose sight is being tested views an eye chart through a trial frame which carries interchangeable lenses. The optometrist selects lenses from a graduated set, interchanging them until the subject has the subjective feeling of optimum visual acuity. The procedure is typically performed for each eye separately, and the overall result is checked by the subject using both eyes.

In its most widely used format, the lenses are interchanged manually by the optometrist. An instrument, known as a phoropter, manufactured by the Moeller-Wedel Company of Germany, in which the lenses are interchanged by means of an electro-mechanical lens changer, is also available, so that the vision test becomes more automated. However, in all of these test procedures, the test is essentially performed stepwise, with the steps being determined by the optometrist, and the subject having to decide subjectively which step provides the best vision correction for his eyes.

A disadvantage of these subjective prior art vision testers is that some subjects find it difficult to determine the optimum correction lens combination because of the stepwise manner in which the lens combinations are changed. This difficulty may be further compounded by the fact that the steps are being made by a person other than the subject, so that the subject does not have any sense of interactive feedback to assist him in determining when optimum visual acuity is attained. An additional factor which may add to this difficulty is the possible presence of a blank step between lens combinations, if the step being executed is not one which involves the simple addition or removal of one component of the lens combination in the frame.

An additional method of testing vision is based on the measurement of the state of refraction of the subject's eye. The ophthalmic refractometer is used for performing this measurement. This method is unlike the trial frame method mentioned above, in that it is an objective measurement which requires no subject intervention at all. However, this type of measurement also has a disadvantage, as will be mentioned below, since objective measurements do not always produce the optimum correction for every subject.

A further disadvantage of the above-mentioned prior art vision testing methods, whether based on the trial frame method or the refractometer method, is that they are only used to correct for the dominant vision defects of defocus and astigmatism, and to a lesser extent, also tilt. Since correction of these low order aberrations generally improves vision to an acceptable level, there has historically been little progress in attempting to test or correct any of the higher order aberrations present in the eye, such as spherical aberration, coma, and the even higher order aberrations, collectively known as irregular aberrations. The term “high order” or “higher order” aberrations, as used throughout this specification and as claimed, is meant to include all those aberrations besides the commonly corrected tilt, defocus and astigmatism aberrations. Furthermore, throughout this specification, and as claimed, the order by which aberrations are referred to are the orders of the wavefront aberrations, as expressed by their Zernike polynomial representation, rather than the order of ray aberrations. Under this convention, tilt, for instance is a first order aberration, defocus and astigmatism are second order aberrations, coma and coma-like aberrations are of third order, spherical and spherical-like aberrations are fourth order, and the above-mentioned irregular aberrations are those of fifth order and higher.

Once the diffraction limit of the eye's imaging capability has been exceeded, this occurring when the pupil size is typically larger than approximately 2 to 3 mm, the size of the minimum detail in the image projected onto the retina, and hence the ultimate visual acuity of the subject, becomes a function of how well the sum total of the aberrations present are corrected. If, in addition to the usually corrected power and astigmatism, higher order aberrations were also corrected, it would be possible to provide super-normal vision for the subject, with performance noticeably better than the commonly accepted optimum vision known as 20/20 vision.

In physical systems, the problem of aberration correction in imaging lenses is solvable by the use of optical design programs, using iterative ray tracing through the lens. But in the case of an in vivo eye, the problem becomes very difficult to solve using conventional optical design technique, both because of the difficulty of defining the true optical parameters of the eye, and also because of the dynamic nature of these parameters in a living eye.

A method whereby higher order aberrations, beyond those of defocus and astigmatism can be measured in the living eye, is described in U.S. Pat. Nos. 5,777,719 and 5,949,719 to D. W. Williams et al, hereby incorporated by reference, each in its entirety. These patents describe the use of a Hartmann-Shack wavefront sensor in combination with a deformable mirror, to compensate for the wavefront distortion introduced by the eye's aberrations. The level of compensation required is a measure of the eye's aberrations, and can be expressed as a correction function consisting of a high order polynomial series. This correction function can be used either to manufacture vision correction lenses according to the aberrations measured, and thus to provide improved vision and even supernormal vision, or to define a correction profile to be ablated by means of laser refractive surgery of the eye, and thus also to improve the vision from that eye.

In the method and apparatus of the above-mentioned Williams et al patents, a laser beam with an incident planar wavefront is projected into the eye, where it is focused onto the retina by the eye's imaging system, and the wavefront emerging from the eye after reflection off the retina is measured. The method is thus an objective optical measurement, in which the aberrations produced by the eye are measured analytically, without involving the subject's perception of the quality of his vision.

Such objective measurement methods, as mentioned above, may have a distinct disadvantage when used to determine vision correction lenses, or vision correction laser refractive surgery. In most cases, the analytically measured aberration corrections do indeed result in optimally corrected vision, as perceived subjectively by the person whose vision is to be corrected. It has been found in statistical studies, however, that in a not insignificant percentage of subjects, the objective optimal vision correction as measured, does not coincide with the subjective feelings of optimal vision by the subject. In these cases, an objective method, whether by refractometer or by the above-mentioned wavefront compensation apparatus, is thus of limited usefulness. There is even an approach accepted among some ophthalmic practitioners which asserts that, on condition that the subject can perform the method reliably, the only valid vision correction methods are subjective ones, since it is a subjective attribute of the subject that is being corrected.

In both of the above-mentioned U.S. patents, it is explicitly stated that “subjective refractive methods of optometrists and objective autorefractors measure defocus and astigmatism only. They cannot measure the complete wave aberration of the eye, which includes all aberrations left uncorrected by conventional spectacles.” In the light of the possible problematic nature of objective testing, as mentioned above, there therefore exists a serious need for an apparatus and method for optometric vision testing, which will provide correction for aberrations higher than the usually measured defocus and astigmatism corrections, and which will provide such measurements according to the subjective perception of the person whose eyes are being tested. In addition, there is also need to provide stepless measurement of the required correction lenses, preferably actuated by the subject himself, in order to facilitate his choice of the best correction lenses.

The disclosures of each of the publications mentioned in this section, and in other sections of the specification, are hereby incorporated by reference, each in its entirety.

### SUMMARY OF THE INVENTION

The present invention seeks to provide a new method and apparatus for optometric vision testing, which overcomes drawbacks and disadvantages of currently available methods and apparatus.

There is thus provided in accordance with a preferred embodiment of the present invention, an optometric vision tester which provides a specification for vision correction according to the optimum visual acuity subjectively perceived by the subject, and which is also able to provide correction for aberrations of higher order than the generally corrected tilt, defocus and astigmatism.

In addition, the apparatus and method of the present invention is able to provide a measurement of the correction required on a continuously variable scale, such that the subject does not have to decide between incremental steps of different correction level, often with the difficulty of a blank intervening stage between each correction step.

Furthermore, the present invention enables the subject to interactively determine when optimum vision has been achieved in the test.

A combination of all of the four above-mentioned advantages of the present invention, namely subjective perception, high order aberration correction, continuously variable adjustment and interactive determination, should lead to the ability to specify the optimum vision correction subjectively attainable, though any combination of at least two of the advantages should preferably provide subjective improvements over other prior art methods.

The specification for vision correction can be either in the form of a prescription for vision correction lenses, whether contact, intra-ocular or spectacle lenses, or in the form of the required corneal profile for vision correction laser refractive surgery. It is to be understood that throughout this specification and as claimed, the term vision correction lens is alternatively used to describe spectacle lenses, contact lenses, or intra-ocular lenses.

Unlike the methods described in the above-mentioned Williams patents, which use a collimated laser beam, the present invention preferably uses a real object, such as an eye chart, effectively at infinity, to provide a planar wavefront incident on the eye's lens system. Additionally, unlike the above-mentioned methods of Williams et al, the best visual acuity of the object as imaged by the eye's lensing system, is determined subjectively by the person whose vision is being tested.

According to a preferred embodiment of the present invention, an optical element with variable and controllable spatial phase properties is located in the optical path of the wavefront, and operates thereon to cancel out aberrations induced into the wavefront by imperfections in the eye's imaging ability. The element does this by introducing a spatially variable phase shift, which is adjusted to be the exact inverse of the phase shift induced into the wavefront reflected from the eye as a result of the aberrations induced by the eye's imaging system. The element can operate on the wavefront either by transmission of the wavefront through it, or by reflection from its surface. Optimum subjective correction of the aberrations is obtained when the subject under test determines subjectively that optimum visual acuity has been achieved. Reading of the settings of the variable element allow the vision correction lenses to be manufactured accordingly, or the laser vision correction profile to be defined accordingly.

If the controllable phase optical element is continuously variable, then the optometric measurement of the eye can be performed on a continuously variable basis, such that easy and positive subject perception of the best visual acuity point may be obtained.

Furthermore, if the subject himself is allowed to control the settings of the variable optical element, a high level of perceptive feedback about the position of optimum visual acuity is obtained, thereby greatly increasing the accuracy of the vision test.

The forms of aberration for which compensation may be made are a function of the types of variable optical element available. Thus, in order to compensate for simple defocus, which is second order aberration, a spherical element with a variable radius of curvature to vary the power is sufficient. The subject varies the radius of curvature until the clearest view of the object is obtained.

In order to compensate for astigmatism, which is another second order aberration, a composite element consisting of two elements of variable power, aligned in two orthogonal directions may be used. The subject varies the power of the elements independently until the optimum visual acuity of the object is perceived in two orthogonal directions. In order to facilitate this adjustment, the object may be provided with variably spaced gratings in orthogonal directions so that the optimum combination can be easily achieved.

In order to compensate for coma and spherical aberration, which are respectively third and fourth order aberrations, a combination of two mutually displaceable aspheric optical elements may be used. Details of how to calculate these elements are given in the section below with detailed descriptions of preferred embodiments. It is to be understood though, that such a combination may also be used for correcting lower order aberrations also.

Alternatively and preferably, instead of elements with specific geometrical optical properties, a single or composite generalized variable optical element may be provided such that compensation may be made for any aberration, regardless of its order, magnitude or symmetry. Such an element may preferably be provided by use of an adaptive optics element or a spatial light phase modulator, such as a deformable mirror, a liquid crystal device, a micro-machined mirror, such as a DMD pixeilated mirror device, or any other suitable optical element capable of being shaped variably and controllably to control the phase of a wavefront.

The electronic control input to the variable phase element is preferably supplied by a computing system, and the signal for the aberration to be compensated is generated as a Zernike polynomial function of order according to the order of aberration. Thus, for compensation of defocus and astigmatism, a second order polynomial function is sufficient. For compensating spherical aberrations, a third order polynomial is needed. For coma, a fourth order polynomial is required., while for the even higher order irregular aberrations, higher order polynomials may be used. The limit of the complexity of the aberration to be compensated is limited only by the ability to provide a suitable control signal which the subject can interactively vary in a systematic manner, so that the compensation which he applies to the element has a clear and unique convergence, to provide optimum vision, free of each compensated aberration. This procedure is facilitated by the fact that the various orders of Zernike polynomial functions operate independently of each other, and that each may be applied separately without affecting the correction for another order aberration, achieved by use of another order polynomial. It is thus possible to formulate a serial correction process, wherein each aberration is corrected separately and sequentially by use of the correct spatial phase shift function for that aberration. In order to correct any residual interaction effects, it is generally advisable to repeat this sequential correction procedure once or twice, to obtain the best iterative correction possible.

There is thus provided, in accordance with a preferred embodiment of present invention, an optometric measurement system which determines the correction lens required to provide optimal visual acuity, the measurement being adjusted interactively by the subject according to his visual perception and which compensates for high order aberrations in the subject's eye.

In accordance with yet another preferred embodiment of the present invention, there is provided a system for determining with essentially continuous variability, wave aberrations originating in an eye of a subject, according to the visual perception of the subject. The essentially continuous variability may be controlled by the subject.

There is further provided in accordance with yet another preferred embodiment of the present invention, a system as described above, wherein the wave aberrations are used in order to determine vision correction data for the eye. The vision correction data may be used for manufacturing a vision correction lens for the eye, or for performing laser refractive surgery on the eye.

In accordance with still another preferred embodiment of the present invention, there is provided a system as described above, wherein the wave aberrations comprise high order wave aberrations, which could be at least third order aberrations.

There is further provided in accordance with still another preferred embodiment of the present invention, a system for correction in an essentially continuously variable manner, of at least one of defocus and astigmatism aberrations originating in an eye of a subject, wherein the correction is performed by the subject according to his visual perception of the subject. The system may also correct higher order wave aberrations originating in the eye of the subject.

In accordance with a further preferred embodiment of the present invention, there is also provided a system for determining high order wave aberrations originating in an eye of a subject, according to the visual perception of the subject. This determining may be controlled by the subject according to the visual perception of the subject. The high order aberrations may be at least the order aberrations, but the system may also determine low order aberrations originating in the eye.

There is provided in accordance with yet a further preferred embodiment of the present invention, a system as previously described, and wherein the aberrations are used in order to determining vision correction data for the subject's vision. The vision correction data may be used for manufacturing a vision correction lens for the eye, or for performing laser refractive surgery on the

There is even further provided in accordance with another preferred embodiment of the present invention, a system for determining vision correction data for an eye of a subject according to the visual perception of the subject, the data providing correction for high order aberrations originating in the eye of the subject. Furthermore, in accordance with yet another preferred embodiment of the present invention, the data may be utilized to manufacture a vision correction lens for the eye, or for performing laser refractive surgery on the eye. The system preferably provides subjective optimal visual acuity for the subject.

There is also provided in accordance with a further preferred embodiment of the present invention, a system for determining vision correction data for an eye of a subject consisting of an object to be viewed by the subject, and an adaptive optical element for adjustment according to the subject's visual perception of the object. The adjustment may be performed by the subject, and may be essentially continuously variable.

In accordance with yet more preferred embodiments of the present invention, the adaptive optical element may be reflective, transmissive, a spatial light modulator, a deformable mirror, a pixellated digital mirror device, or a liquid crystal device. Furthermore, the adaptive optical element may preferably consist of at least two juxtaposed optical plates having preselected profiles, and wherein the adjustment is performed by mutual motion of the plates.

There is further provided in accordance with yet another preferred embodiment of the present invention, a system as described above, and wherein the vision correction data provides correction for high order aberration originating in the eye. This vision correction data may be used for manufacturing a vision correction lens for the eye, or to perform laser refractive surgery on the eye.

In accordance with still another preferred embodiment of the present invention, there is provided a method for determining wave aberrations originating in an eye of a subject, consisting of the steps of providing an object to be viewed by the subject, inserting an adaptive optical element into the optical path between the eye and the object, and allowing the subject to adjust the element to achieve optimum visual perception of the object. The method may also preferably consist of the step of using the wave aberrations to determine vision correction data for the eye, and the vision correction data may be used for manufacturing a vision correction lens for the eye, or for performing laser refractive surgery on the eye.

There is further provided in accordance with still another preferred embodiment of the present invention, a method as described above, wherein the wave aberrations comprise high order wave aberrations.

In accordance with a further preferred embodiment of the present invention, there is also provided a method as described above, wherein the subject adjusts the element in an essentially continuously variable manner.

There is also provided in accordance with yet further preferred embodiments of the present invention, a method as described above, wherein the adaptive optical element may be reflective, transmissive, a spatial light modulator, a deformable mirror, a pixellated digital mirror device, or a liquid crystal device.

There is even further provided in accordance with a preferred embodiment of the present invention, a method as described above, wherein the adaptive optical element consists of at least two juxtaposed optical plates having preselected profiles, and wherein the adjustment is performed by mutual motion of the plates.

### BRIEF DESCRIPTION OF THE DRAWINGS

The present invention will be understood and appreciated more fully from the following detailed description, taken in conjunction with the drawings in which:

FIG. 1 is a schematic view of a prior art method of vision testing using a trial frame;

FIG. 2 is a schematic diagram of a system for vision testing according to a preferred embodiment of the present invention, using a transmissive adaptive optical element;

FIG. 3 is a schematic illustration of a vision correction element according to another preferred embodiment of the present invention, as used in the system shown in FIG. 2, but made up of a fixed lens in combination with an adaptive optical element;

FIG. 4 is a schematic diagram of a system for vision testing according to another preferred embodiment of the present invention, using a reflective adaptive optical element;

FIG. 5 is a view of a deformable mirror, such as could be used in the of FIG. 4, showing how deformations are introduced according to the symmetry and order of the correction polynomial required;

FIG. 6A, FIG. 6B and FIG. 6C are schematic illustrations of the use of mechanically adjustable adaptive optical elements for aberration compensation, according to other preferred embodiments of the present invention; FIGS. 6A and 6B show the use of an aspheric refractive element, while FIG. 6C shows an element resembling a section of the corridor of a progressive lens, whose motion changes the spherical power in the optical path;

FIGS. 7A and 7B are drawings of an adaptive optical element whose radiu of curvature is adjustable by means of a change in the internal pressure within the element;

FIGS. 8A to 8C are drawings showing three positions of a set of prior art Alvarez-Humphries plates, whose spherical power can be adjusted according to their mutual position;

FIG. 9 is a schematic diagram of the use of a pair of Palusinsky plates in order to introduce a known compensating aberration, according to another preferred embodiment of the present invention; and

FIG. 10 is a schematic illustration of an interactive adaptive optical system, according to yet another preferred embodiment of the present invention, for correcting defocus and astigmatism.

### DETAILED DESCRIPTION OF PREFERRED EMBODIMENTS

Reference is now made to FIG. 1, which illustrates schematically a prior art method of vision testing using a trial frame. The subject 10 views the test card 12, located at an optically effectively infinite distance from him, through a pair of trial frames 14, into which the optometrist can insert combinations of lenses 16 until the best visual acuity is obtained for the subject. The lens combination is changed incrementally until the best combination is determined, as per the subjective perception of the person 10 whose sight is being tested. Such a trial frame is generally used for correcting defocus, by the use of combinations of spherical lenses, and astigmatism, by the addition of cylindrical lenses, mutually rotated.

Though probably the most widely used method in many parts of the world, this method has three disadvantages (i) the changes in correction lens are incremental, which in some cases makes it more difficult for the subject to determine the optimum combination; (ii) it is operated by the optometrist, which adds to the subject's uncertainty of the optimum combination; and (iii) it is not generally used to correct aberrations other than simple defocus, astigmatism and tilt.

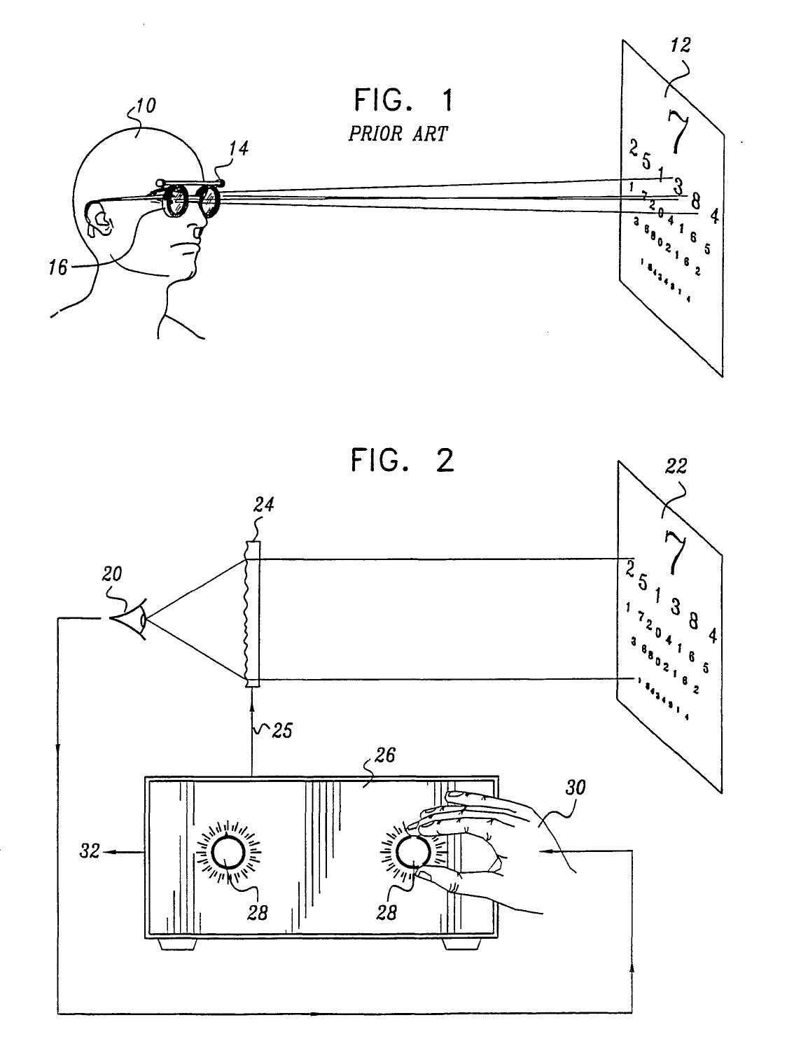

Reference is now made to FIG. 2, which is a schematic diagram of a system for vision testing according to a preferred embodiment of the present invention, using an adaptive optical element. As with the prior art trial frame method, the subject whose vision is under test views the test card 22, located at an optically effective infinite distance from his eye 20. Instead of the trial frame, a transmissive adaptive optical element 24 is located in front of the subject's eye, and the test card is viewed through it. The transmissive adaptive optical element could preferably be a liquid crystal element, which introduces a phase shift into the wavefront which can be spatially modulated according to the individual voltage signals applied to each of the pixels in the LC element. The electronic control inputs 25 to the adaptive optical element 24 enable the individual pixels of the element to change their phase delay according to the spatial pattern generated by the control unit 26. The spatial geometry of the element is determined by the settings of the control knobs 28, which can be adjusted by the subject's hand 30, to provide the best visual acuity of the test card. When the optimum settings have been attained, these settings are output 32 from the control unit 26, preferably in a form that enables the correction lens to be manufactured according to those settings, or that enables the laser refractive surgical treatment to be executed according to those settings.

In order to correct defocus, the adaptive optical element need only be capable of changing its spherical power. In order to correct astigmatism, the numerical test card shown is replaced with a test card which incorporates orthogonally aligned visual acuity and resolution patterns, so that it is easy for the subject to determine when the level of astigmatism present has been optimally compensated using the controls 28 on the control unit 26. The adaptive optical element is adjusted by means of the control unit to add a graduated level of cylinder to the lens, with the axis of the cylinder aligned according to the subject's own perception of his view of the test card.

FIG. 3 schematically shows a further preferred embodiment of the present invention, in which the vision correction element is made up of a conventional lens 40 in combination with an adaptive optical element 42. This embodiment is preferably used in order to simplify the operational requirements demanded of the adaptive optical element. Such elements typically have a limited range of adjustment. In the embodiment shown in FIG. 3, the conventional lens 40, which may be interchangeable, may be preferably used to correct the lower order aberrations such as defocus and astigmatism, while the higher order aberrations, which may be only a small perturbation on the complete lens performance, are corrected interactively by means of the input control signals 44 to the adaptive optical element 42. According to vet further preferred embodiments of the present invention, the adaptive optical element may also take part of the fixed refractive work of the correction lens, such that minor corrections of power can be undertaken interactively, as well as the higher order aberration corrections.

According to yet a further preferred embodiment of the present invention, the conventional element 40 may be determined by an initial objective test of the subject's vision, such as by using a refractometer, or by a prior art subjective measurement, such as the trial frame method described in the background section of this application, and this element is assumed to represent a first order vision-correction step. The adaptive optical element 42 is then used in order to fine-tune the vision correction according to the subject's subjective perception of his best vision. This fine-tuning can be limited to additional fine correction of the low order aberrations approximately corrected by the initially chosen conventional element, or it can include also fine corrections for higher order aberrations, which are not even addressed in the initial objective test.

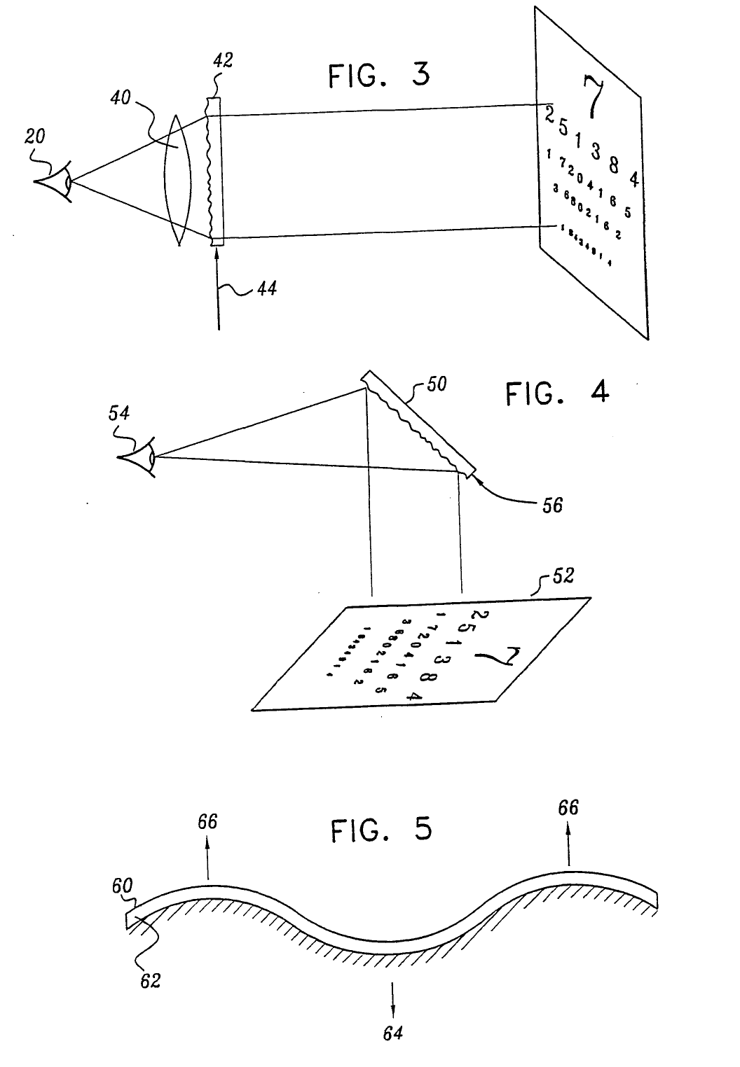

Reference is now made to FIG. 4, which is a schematic diagram of a system for vision testing according to yet another preferred embodiment of the present invention, using a reflective adaptive optical element 50, which reflects the light from the test card into the subject's eye 54. As previously, the subject interactively controls the element by means of control signals 56, adjustment being made according to the subject's perception of optimum visual acuity of the test card. This embodiment has an advantage over the transmissive embodiment shown in FIG. 2 in that there is a wider range of adaptive elements available for reflective use than for transmissive use. The reflective element 50 may preferably be a deformable mirror, or a pixellated digital mirror device. The actuators of each spatially separate part of the adaptive optical element are controlled by the subject-operated control system.

Reference is now made to FIG. 5 which is a view of a deformable mirror, such as could be used in the system of FIG. 4, showing how deformations are programmed according to the symmetry and order of the correction polynomial required. The top surface 60 of the deformable mirror 62 shown in FIG. 5 is the reflective surface. In FIG. 5, the actuators have been programmed to provide a concave profile to the reflector at the center 64, and a convex profile at the extremities 66. The arrows are schematic indications of the direction of deformation. Though only three arrows are shown, it is to be understood that in a real deformable mirror, the number of actuated pixels is many times higher than this. In the case of a DMD device, the number can run into tens of thousands.

The simple symmetry shown in the mirror of FIG. 5, if circularly symmetric, is suitable for correcting aberrations of spherical symmetry, such as the spherical aberrations. In order to correct astigmatism and coma, a deformation pattern with linear symmetry across the width of the mirror is needed. In general, when deformable mirrors such as that shown in FIG. 5, or other SLM's are used, the deformations are computer generated according to the Zernike polynomials, and the amount of the deformation is controlled by the subject. The adjustment commences with the low order corrections and progresses to the higher orders. The process is preferably repeated twice in order to iteratively achieve the best solution, as previously mentioned. Control of the adjustments themselves may preferably be achieved by any of the commonly used computer control input devices, such as a joystick which may preferably be used for adjusting the intensity of the terms of the correction polynomial, with a pushbutton for switching orders.

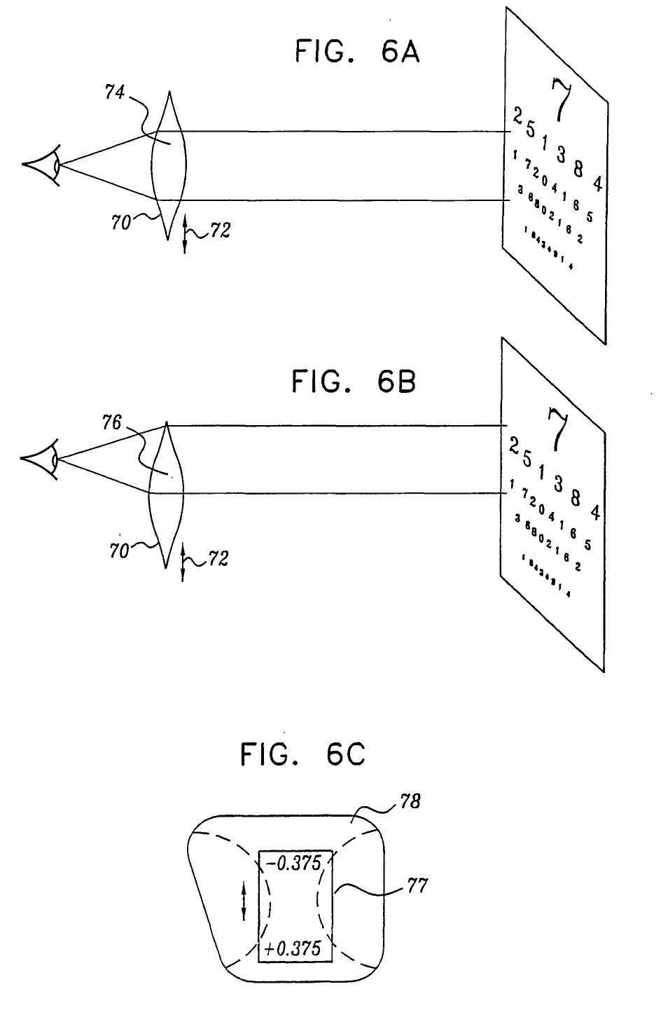

Reference is now made to FIGS. 6A to 6C, which are schematic illustrations of yet further preferred embodiments of the present invention, wherein the optical element is adaptive since it can be adjusted by the user, but wherein this adjustment is performed mechanically. In FIGS. 6A and 6B is shown an aspheric element 70, which is shown as a refractive element, but could equally be reflective, with a variable radius of curvature as a function of spatial position on the element. By moving the element, either by a translation 72, or by a non-axial rotation, different radii of curvatures are inserted into the optical path between the subject's eye and the test plate, and the subject's vision correction is thereby changed on a continuous basis. This variable radius of curvature element is thus suitable for correcting defocus defects in the subject's vision. In FIG. 6A, the light is seen traversing the center of the lens 74, having a strong corrective power, while in FIG. 6B, the element has been traversed mechanically such that the light passes through the lens at a distance from the axis 76, which is of lower power. The position of the element can be calibrated in terms of its focal correcting power, such that as the user moves the element to the position of optimum visual acuity, the value of the correcting power can be determined from the lateral position of the element. Though in FIGS. 6A and 6B, the subject's eye is shown in different positions in order to illustrate the different powers obtained, in practice, the subject's eye would be at a fixed position, and the different powers used to provide different levels of vision correction. The slight additional prism introduced by the use of the lens non-paraxially can generally be neglected, especially if the lens is used to fine tune the aberration correction, using an element obtained by means of an objective measurement to approximately correct the aberration, as described in the embodiment of FIG. 3.

In FIG. 6C is shown another preferred embodiment similar to that shown in FIGS. 6A and 6B, wherein the single element 77 moved by the subject to change the corrective power resembles a section cut from the corridor of a progressive spectacle lens, shown virtually in dotted outline 78. At one end of the element the power in the example shown is +0.375, and at the other it is −0.375. Lateral motion of the element thus covers that range of corrective spherical power.

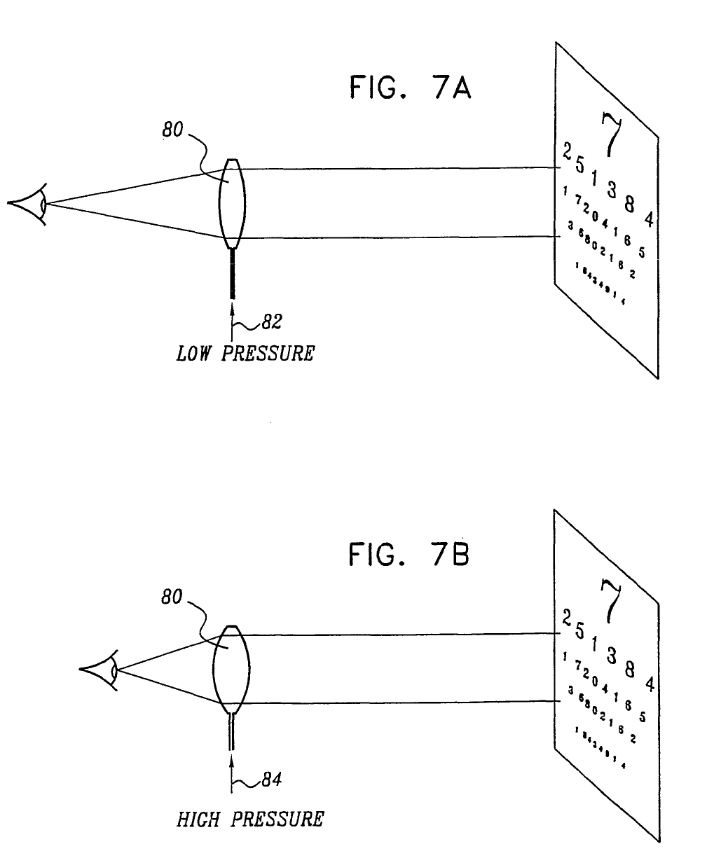

According to yet another preferred embodiment of the present invention, the element may be such that it has a uniform radius of curvature, which is adjustable by the provision of hydraulic or pneumatic pressure within the element, such that control of the power is effected by the internal pressure applied. Such an element 80 is shown in a refractive form in FIGS. 7A and 7B, though it is clear to one skilled in the art that a reflective form may be likewise constructed. In FIG. 7A, a low internal pressure 82 is applied, providing a weak spherical power correction, whereas in FIG. 7B, a high internal pressure 84 is applied, providing strong spherical power correction.

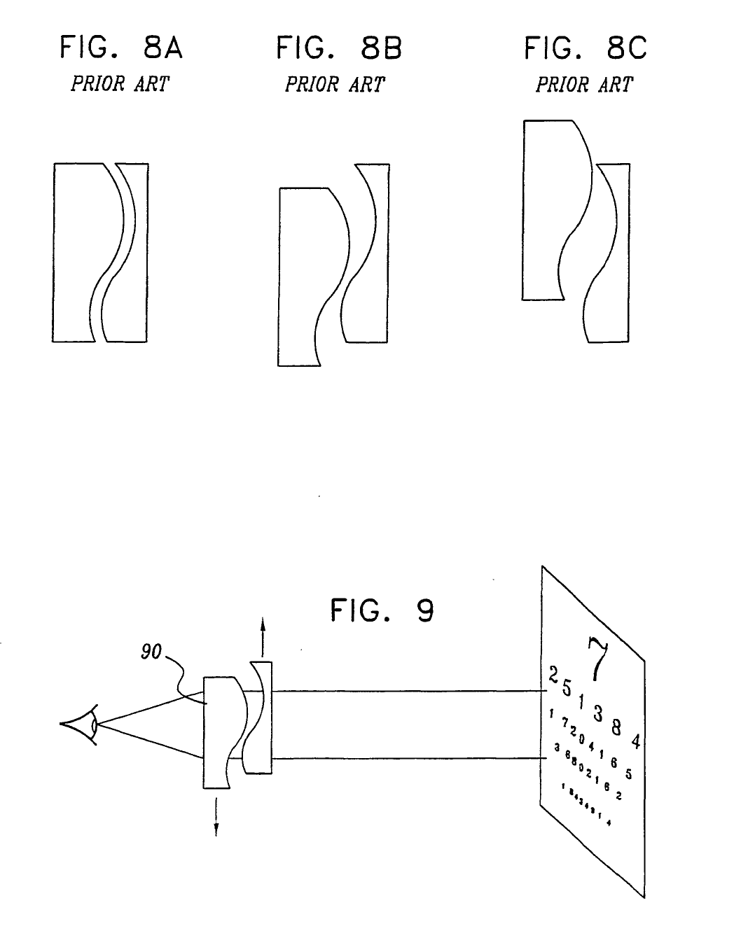

Reference is now made to FIGS. 5A to 8C, which are illustrations of a pair of Alvarez-Humphrey plates, as described in U.S. Pat. No. 3,507,565 for a “Variable power lens and system”, hereby incorporated in its entirety by reference. This combination of superimposed cubic aspheric plates generates a variable amount of optical power when the places are laterally moved relatively to each other, ranging from a neutral plate as shown in the neutral position in FIG. 8A, to a positive lens, as shown in FIG. 8B, and to a negative lens, as shown in FIG. 8C, depending on the direction of the lateral mutual shift of the plates. Such prior art plates can be used, according to a further preferred embodiment, to subjectively correct for defocus using the methods of the present invention.

In an article entitled “Lateral-shift variable aberration generators” published by I. A. Palusinsky et al., in Applied Optics Vol. 38 No.1, pp. 86-90 (January 1999), hereby incorporated in its entirety by reference, there is described a method of extending the Alvarez-Humphrey plate design to include elements capable of generating wavefront aberrations of higher order than only the defocus considered by Alvarez and Humphrey. In that article, there are given analytical formulae for calculating the plate surface profiles required to introduce variable amounts of the common aberrations up to fourth order, including tilt, defocus, astigmatism, coma, spherical aberration and line coma. Reference is especially made to Table 1 of the Palusinski et al article, and its accompanying text. Several surface profiles may be superimposed in one pair of plates, thus simultaneously generating a combination of aberrations, but with a fixed ratio between them.

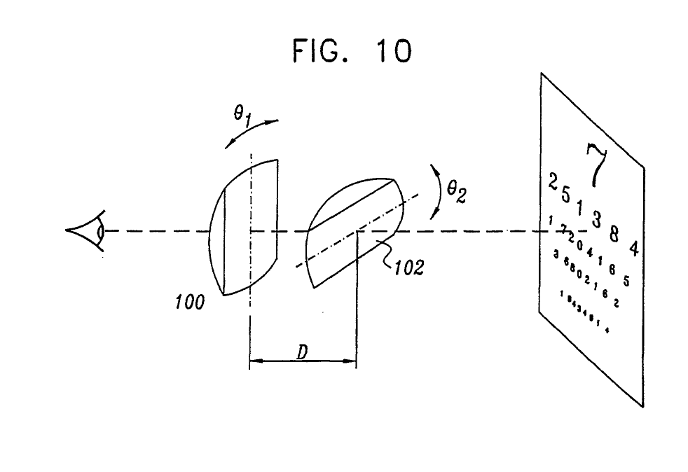

FIG. 9 is a schematic illustration of how such plate pairs 90 may be utilized, according to more preferred embodiments of the present invention, as the variable adaptive optical element in the apparatus and method of the present invention. Plate pairs with different surface profile are chosen, preferably according to the formulae given in the Palusinsky et al article, to compensate for the various different aberrations required to be taken into account. In order to compensate for each aberration independently, a separate pair of plates is generally required for each aberration type.

Reference is now made to FIG. 10, which is a schematic illustration of a further preferred embodiment of the present invention, operative to correct low order aberrations. The variable refractive correction element is composed of a pair of cylindrical lenses 100, 102, mounted such that the distance D between them may be controllably varied, and their angular alignments, θ₁ and θ₂ may also be controllably varied. Variation of the distance D between them results in a change in the power of the doublet, thereby correcting for defocus in the subject's eye, while mutual rotation of one with respect to the other results in a change in the net cylinder and its axis, such that astigmatism can thus be corrected. In FIG. 10, the cylindrical nature of the lenses has been purposely exaggerated, in order to illustrate the way in which this preferred embodiment is constructed and operates.

A common feature of all of the above mechanically adaptive optical elements is that the subject is able to control their optical corrective power monotonically and in such a way as to enable him to subjectively determine the position of optimum correction. The position can then be converted, preferably by means of an electromechanical interface device consisting of linear and angular encoders, to provide prescription information as to the corrective optic required for each of that particular subject's eyes.

It will be appreciated by persons skilled in the art that the present invention is not limited by what has been particularly shown and described hereinabove. Rather the scope of the present invention includes both combinations and subcombinations of various features described hereinabove as well as variations and modifications thereto which would occur to a person of skill in the art upon reading the above description and which are not in the prior art.

We claim:

1. A system for determining with essentially continuous variability, wave aberrations originating in an eye of a subject, according to the visual perception of said subject.

2. A system according to claim 1, wherein said essentially continuous variability is controlled by said subject.

3. A system according to either of claim 1 and claim 2, wherein said wave aberrations are used in order to determine vision correction data for said eye.

4. A system according to claim 3, and wherein said vision correction data are used for manufacturing a vision correction lens for said eye.

5. A system according to claim 3, and wherein said vision correction data are used for performing laser refractive surgery on said eye.

6. A system according to any of claim 1 to claim 5, wherein said wave aberrations comprise high order wave aberrations.

7. A system according to claim 6, wherein said high order aberrations are at least third order aberrations.

8. A system for correcting with essentially continuous variability at least one of defocus and astigmatism aberrations originating in an eye of a subject, wherein said correcting is performed by said subject according to the visual perception of said subject.

9. A system according to claim 8, and which also corrects higher order wave aberrations originating in said eye of said subject.

10. A system for determining high order wave aberrations originating in an eye of a subject, according to the visual perception of said subject.

11. A system according to claim 10, wherein said determining is controlled by said subject according to said visual perception of said subject.

12. A system according to claim 10, wherein said high order aberrations are at least third order aberrations.

13. A system according to claim 10, and wherein said system also determines low order aberrations originating in said eye.

14. A system according to any of claim 10 to claim 13, and wherein said aberrations are used in order to determine vision correction data for said subject's vision.

15. A system according to claim 14, and wherein said vision correction data are used for manufacturing a vision correction lens for said eye.

16. A system according to claim 14, and wherein said vision correction data are used for performing laser refractive surgery on said eye.

17. A system for determining vision correction data for an eye of a subject according to the visual perception of said subject, said data providing correction for high order aberrations originating in said eye of said subject.

18. A system according to claim 17, and wherein said data is utilized to manufacture a vision correction lens for said eye.

19. A system according to claim 17, and wherein said data is utilized to perform laser refractive surgery on said eye.

20. A system according to claim 17, and wherein said system provides subjective optimal visual acuity for said subject.

21. A system for determining vision correction data for an eye of a subject comprising:
an object to be viewed by said subject; and
an adaptive optical element for adjustment according to the subject's visual perception of said object.

22. A system according to claim 21, wherein said adjustment is performed by said subject.

23. A system according to claim 21, wherein said adjustment is essentially continuously variable.

24. A system according to claim 21, wherein said adaptive optical element is reflective.

25. A system according to claim 21, wherein said adaptive optical element is transmissive.

26. A system according to claim 21, wherein said adaptive optical element is a spatial light modulator.

27. A system according to claim 24 wherein said adaptive optical element is a deformable mirror.

28. A system according to claim 24, wherein said adaptive optical element is a pixellated digital mirror device.

29. A system according to claim 25, wherein said adaptive optical element is a liquid crystal device.

30. A system according to claim 25, wherein said adaptive optical element comprises at least two juxtaposed optical plates having preselected profiles, and wherein said adjustment is performed by mutual motion of said plates.

31. A system according to any of claims 21 to 30, and wherein said vision correction data provides correction for high order aberrations originating in said eye.

32. A system according to any of claims 21 to 30, and wherein said vision correction data is used for manufacturing a vision correction lens for said eye.

33. A system according to any of claims 21 to 30, and wherein said vision correction data is used to perform laser refractive surgery on said eye.

34. A method for determining wave aberrations originating in an eye of a subject comprising the steps of:
providing an object to be viewed by said subject;
inserting an adaptive optical element into the optical path between said eye and said object; and
allowing said subject to adjust said element to achieve optimum visual perception of said object.

35. A method according to claim 34, and also comprising the step of using said wave aberrations to determine vision correction data for said eye.

36. A method according to claim 35 and also comprising the step of using said vision correction data for manufacturing a vision correction lens for said eye.

37. A method according to claim 35, and also comprising the step of using said vision correction data for performing laser refractive surgery on said eye.

38. A method according to any of claims 34 to 37, wherein said wave aberrations comprise high order wave aberrations.

39. A method according to any of claims 34 to 38, wherein said subject adjusts said element in an essentially continuously variable manner.

40. A method according to any of claims 34 to 39, wherein said adaptive optical element is reflective.

41. A method according to any of claims 34 to 39, wherein said adaptive optical element is transmissive.

42. A method according to either of claims 40 and 41, wherein said adaptive optical element is a spatial light modulator.

43. A method according to claim 40, wherein said adaptive optical element is a deformable mirror.

44. A method according to claim 40, wherein said adaptive optical element is a pixellated digital mirror device.

45. A method according to claim 41, wherein said adaptive optical element is a liquid crystal device.

46. A method according to claim 41, wherein said adaptive optical element comprises at least two juxtaposed optical plates having preselected profiles, and wherein said adjustment is performed by mutual motion of said plates.
